(19) United States
(12) Patent Application Publication
(10) Pub. No.: US 2010/0298711 A1
(43) Pub. Date: Nov. 25, 2010

(54) WIRELESS ULTRASOUND TRANSDUCER USING ULTRAWIDEBAND

(75) Inventors: Peder C. Pedersen, Sterling, MA (US); Thomas L. Szabo, Newburyport, MA (US); Philip Joseph Cordeiro, Providence, RI (US)

Correspondence Address: ROPES & GRAY LLP, PATENT DOCKETING 39/41, ONE INTERNATIONAL PLACE, BOSTON, MA, 02110-2624, US

(73) Assignee: Worcester Polytechnic Institute, Worcester, MA (US)

(21) Appl. No.: 12/524,933

(22) PCT Filed: Jan. 28, 2008
(86) PCT No.: PCT/US08/01068

§ 371 Date: Jul. 14, 2010

Related U.S. Application Data
(60) Provisional application No. 60/898,343, filed on Jan. 29, 2007.

(51) Int. Cl. A61B 8/14

(52) U.S. Cl. 600/459

(57) ABSTRACT

Ultrasound imaging systems and methods are described that include a cableless front end having a UltraWideBand transceiver and a beamformer that transmit ultrasound data to a back end physically separated from the front end, with the back end having a UltraWideBand transceiver for receiving ultrasound data from the front end UltraWideBand transceiver via a peer-to-peer communication protocol. The back end unit can also receive and transmit commands to and from the front end unit to select an image mode and has a signal processor and scan converter that can convert ultrasound data into image data for display. An input device in the front end allows the operator to selectively display ultrasound data or patient data on an auxiliary worn display or the nearest local display.

### CROSS-REFERENCE RELATED APPLICATION

This application claims the benefit of U.S. Provisional Application Ser. No. 60/898,343, titled “Untethered Ultrawideband Ultrasound Imaging, Information Management and Display,” filed Jan. 29, 2007, the entire contents of which are incorporated herein by reference.

### GOVERNMENT CONTRACT

The U.S. Government has a paid-up license in this invention and the right in limited circumstances to require the patent owner to license others on reasonable terms as provided for by the terms of U.S. Army Medical Research Activity (USAMRAA) Contract No. DAMD17-03-2-0006.

### FIELD OF THE INVENTION

In general, the invention relates to the field of ultrasound imaging for medical and non-destructive testing purposes, particularly ultrasound imaging using UltraWideBand (UWB) technology.

### BACKGROUND OF THE INVENTION

Medical ultrasonography generally utilizes ultrasound technology to image soft tissues of the human body. Common applications in medicine include uses in the specialties of cardiology, endocrinology, gastroenterology, and obstetrics, among others.

Conventional medical ultrasound diagnostic systems can include an ultrasound array transducer, a front-end and a computational back-end. The array transducer can be connected to the front-end using a multi-coaxial wired connection, and RF signals from the front end are transmitted to the back end via appropriate cables while transducer control signals are transmitted from the back end to the front end via separate cables. The front end and the back end are typically placed inside an ultrasound scanner housing, although in some cases, the front end is external to the ultrasound scanner housing.

A drawback to conventional ultrasound systems is the fact that the transducer's range of motion is limited to the length of the coaxial cable connecting it to the front end. This will require the patient to be located within the cable's reach, which can thereby necessitate moving the entire ultrasound system if the patient cannot be moved to within the reach of the coaxial cable.

Using ultrasound systems with components connected by cables can limit or reduce the type, range, and/or safety of available clinical applications. For example, conventional transducers use very long cables for intra-operative procedures, in order to place the imaging system outside the surgical area and to conserve space in the operating room. These cables can be hazardous in the surgery area, because space is at a premium, because they can be a source of contamination, and because they can be a hazard to medical personnel (e.g., can cause them to trip on the wires). Cables can also limit accessibility and freedom of movement to restricted, compartmentalized, hazardous or contaminated areas in which diagnostic ultrasound is needed.

### SUMMARY OF THE INVENTION

It would be desirable to eliminate the use of cables connecting a combined transducer and front end with the back end of an ultrasound system. It would also be desirable to integrate the transducer of an ultrasound system with the front end in a single housing. It would also be desirable to use transducers having large numbers of elements in ultrasound systems without using cables to transmit data to a back end for processing.

The present invention eliminates the need for such cables in ultrasound devices in which a front end is either directly integrated with a transducer array or placed in close proximity to the transducer array. Certain embodiments of invention utilize a UltraWideBand transceiver, to be denoted the front end UltraWideBand transceiver, which is connected to the beamformer output of the front end. The invention further utilizes a second UltraWideBand transceiver, to be denoted the back end UltraWideBand transceiver, which is connected to the back end. The two UltraWideBand transceivers communicate in half or full duplex mode using an UltraWideBand (UWB) wireless protocol, such that the back end UltraWideBand transceiver receives ultrasound data from the front end UltraWideBand transceiver via a peer-to-peer communication protocol, and the front end UltraWideBand transceiver receives control signals from the back end regarding beam forming in transmit and receive modes and other important imaging parameters via a peer-to-peer communication protocol.

Because the transducer and front end can be integrated and miniaturized, the ultrasonographer (or operator) may obtain medical ultrasound images by operating the ultrasound transducer, and the RF data is transmitted to the back-end via the UWB-enabled RF-link. The present invention can be used in a wide variety of applications. For example, the UWB system can be used in a surgical suite, which can be advantageous because there can be fewer cables to interfere with the surgery and potentially contaminate the surgical suite. In one implementation, the UWB system is combined with a microphone and speech recognition system to replace the need for a keyboard, thereby eliminating an additional source of contamination and freeing up additional space. Alternatively, the UltraWideBand system may be combined with a touch screen display to replace the need for a keyboard. The present invention can also be advantageous because the system's weight can be reduced, thereby increasing ease of mobility. Furthermore, the present invention can allow a wider range of displays to be used (e.g., wearable displays and displays affixed to a patient's bed). Also, the present invention can allow computational capabilities to be expanded more easily, and can simplify networking of multiple ultrasound systems.

The UltraWideBand system operates in a duplex mode, to send beam formed RF data from the front end to the back end, while control data is sent from the back end to the front end. In one embodiment, the UWB system operates in full duplex mode, which means that RF signal and control data are being transmitted simultaneously. The full duplex mode enables the operator/sonographer to wear or carry a display, which is wirelessly connected to the back end.

In an alternative embodiment, the UWB system operates in a half duplex mode, where the RF signal is sent from the beamformer of the front end to the back end as the RF signal is being generated. The control data is sent from the back end to the front end in the time intervals where no RF signal is being transmitted. In this embodiment, the front end includes memory for storage of the control signals as well as the processing power to apply the control signals at the proper time instances.

In one embodiment, the peer-to peer communication protocol is an IEEE 1394-conforming protocol (e.g., IEEE 1394 a/b), which allows full duplex operation. In alternative implementations, the peer-to-peer protocol is a USB2 protocol, which allows half duplex operation. In some embodiments, the front end and the beamformer are enclosed in a separate housing, and the front end UltraWideBand transceiver couples to the separate housing via a cable. In another embodiment, the front end with the beamformer is integrated with the front end UltraWideBand transceiver. In yet another embodiment, the array transducer, the front end with the beamformer and the front end UltraWideBand transceiver are all integrated into the transducer housing.

In some embodiments, the front end UltraWideBand transceiver and the back end UltraWideBand transceiver are in communication using an UltraWideBand protocol in duplex mode. In some embodiments, the front end UltraWideBand transceiver and the back end UltraWideBand transceiver are in communication using orthogonal frequency-division multiplexing and transmitting data using the IEEE 1394a/b data transfer standard. Also, in some embodiments, the front end UltraWideBand transceiver and the back end UltraWideBand transceiver can be configured to utilize ECMA-368 UltraWideBand.

Some embodiments include a display, wherein the display can be in communication with the back end. In some embodiments, the display is wearable. In another embodiment, the display is a touch screen display that doubles as an input device. Also, in some embodiments, the back end UltraWideBand transceiver can be in communication with a computer network, wherein the computer network can store a plurality of patient data, and the back end UltraWideBand transceiver can be configured to transmit a selection of the patient data from the computer network to the display. The selected patient data can in some embodiments include a set of stored patient image data. In some embodiments, the selected patient data can include a set of vital data versus time for a patient, the set of vital data versus time selected from the group consisting of blood pressure, heart rate, fluid intake, temperature, and medication. In some embodiments, the display can be in communication with a display UltraWideBand transceiver, the display UltraWideBand transceiver can be in communication with the back end UltraWideBand transceiver, and the display UltraWideBand transceiver can be configured to receive streamed video data from the back end.

In some embodiments, the beamformer can be an analog beamformer, and in other embodiments, the beamformer can be a digital beamformer. In some embodiments, the front end UltraWideBand transceiver and the back end UltraWideBand transceiver can be configured to transmit and receive data in the form of RF beamformed data and commands.

An ultrasound system of the present invention can in some embodiments also include a power source coupled to the front end UltraWideBand transceiver. In some embodiments, the power source may be external to the transducer and the beamformer, and in some other embodiment the power source may be integrated into the transducer along with the beamformer and possibly also the front end UltraWideBand transceiver.

In some embodiments, the back end can be implemented in the form of software. In some embodiments, the back end can be implemented on a local computer. The local computer can be, in some embodiments, configured to access a set of patient data stored at a remote location, and can, in some embodiments, include a display. Alternatively, the back end and back end UltraWideBand transceiver can be integrated into the display unit.

An ultrasound system of the present invention can, in some embodiments, further include a second front end, wherein the second front end can contain a second beamformer and a second front end UltraWideBand transceiver, wherein the second front end can be coupled to a second transducer array, and the second front end transmits ultrasound data processed by the second beamformer to a second back end UltraWideBand transceiver, wherein the second front end UltraWideBand transceiver is in communication with the back end, using the second front end UltraWideBand transceiver. In some embodiments, the back end can be in communication with a computer network via a satellite communication system. In some embodiments, the back end can be integrated into the infrastructure of a mobile vehicle. Such embodiments enable improved telemedicine and emergency response by allowing for on-site ultrasound where it was not previously available.

In some embodiments, an ultrasound system in accordance with the present invention can further include a robotic arm, wherein the transducer array can be coupled to the robotic arm, a control device for directing the robotic arm, and a display for displaying the image data. In some embodiments of this system, a second robotic arm in communication with the control device, employs at least one surgical tool coupled to the second robotic arm.

In some embodiments, a display in communication with the back end for displaying the image data is centrally mounted in a surgical suite. Also, in some embodiments, a control apparatus for accepting commands to control the ultrasound system can be included. The commands can include, for example, imaging commands in some embodiments, including imaging mode, range, focus depth, time/gain control setting.

In some embodiments, the control apparatus is coupled to the front end and is in communication with the back end. Specifically, the front end UltraWideBand transceiver is configured to transmit the imaging commands to the back end UltraWideBand transceiver, the back end is configured to convert the imaging commands into a set of instructions for the front end, and the back end UltraWideBand transceiver is configured to transmit the set of instructions to the front end through the front end UltraWideBand transceiver. In some embodiments, the commands can be acoustic field generating commands, selected from the group including commands for frame rate, focal points, range, active elements, and scan line repeat for Doppler. In other embodiments, imaging commands can be translated directly into front end instructions within the front end via a separate processor included therein.

In some embodiments, the control apparatus can be coupled to the back end and be in communication with the front end and the back end UltraWideBand transceiver can be configured to transmit the acoustic field generating commands to the front end through the front end UltraWideBand transceiver. In some embodiments, a display is in communication with the control apparatus. In such embodiments, the commands include display commands. The display commands may include, for example, commands to retrieve patient data. In some embodiments, the control apparatus can be a microphone in communication with a speech recognition system to accept voice commands to control the ultrasound system. In another embodiment, a touch screen display is used, in which case the display and the control apparatus is combined into one device.

In some embodiments, an encryption module for encrypting data transferred by at least one of the front end or back end UltraWideBand transceivers can be included.

In one embodiment, the UltraWideBand system also includes a plurality of locations with each location including a respective display and at least one back end UltraWideBand transceiver capable of communicating with one or more front end UltraWideBand transceivers. In one embodiment, there is only one display associated with one back end UltraWideBand transceiver, and in such a case the selection of the display is automatic. In another embodiment, there are two or more displays associated with one back end UltraWideBand transceiver, and in such a case the display to be used is selected manually or based on data transmitted to the back end UltraWideBand transceiver along with the beamformed ultrasound data.

The front end includes a beamformer and a front end UltraWideBand transceiver, coupled to the beamformer, wherein the beamformer can provide ultrasound data corresponding to a selected (automatically or manually) display. The back end contains a back end UltraWideBand transceiver for receiving RF data from the front end UltraWideBand transceiver, via a peer-to-peer communication protocol, and includes a signal processor circuit, wherein the signal processor converts the received RF ultrasound data into image data to be directed to the selected (automatically or manually) display.

### BRIEF DESCRIPTION OF THE DRAWINGS

The above and other advantages of the present invention will become more apparent upon consideration of the following detailed description, taken in conjunction with the accompanying drawings, in which like reference characters refer to like parts throughout, and in which:

FIG. 1 is a schematic diagram of a system for ultrasound imaging according to an illustrative embodiment of the invention;

FIG. 2 is a schematic diagram of a system for ultrasound imaging according to an illustrative embodiment of the invention;

FIG. 3 is a flowchart of a method for using an ultrasound imaging system according to an illustrative embodiment of the invention;

FIG. 4 is a schematic diagram of a system for ultrasound imaging according to an illustrative embodiment of the invention;

FIG. 5 is a flowchart of a method for retrieving patient information from an information network according to an illustrative embodiment of the invention;

FIG. 6 is a schematic diagram of a system for ultrasound imaging in a mobile vehicle application according to an illustrative embodiment of the invention;

FIG. 7 is a schematic diagram of a system for remotely guided surgery using ultrasound imaging according to an illustrative embodiment of the invention; and

FIG. 8 is a flowchart of a method for performing remotely guided surgery using an ultrasound imaging system according to an illustrative embodiment of the invention.

### DETAILED DESCRIPTION OF THE INVENTION

To provide an overall understanding of the invention, certain illustrative embodiments will now be described, including systems and methods for ultrasound imaging. However, it will be understood by one of ordinary skill in the art that the systems and methods described herein may be adapted and modified as is appropriate for the application being addressed and that the systems and methods described herein may be employed in other suitable applications, and that such other additions and modifications will not depart from the scope hereof.

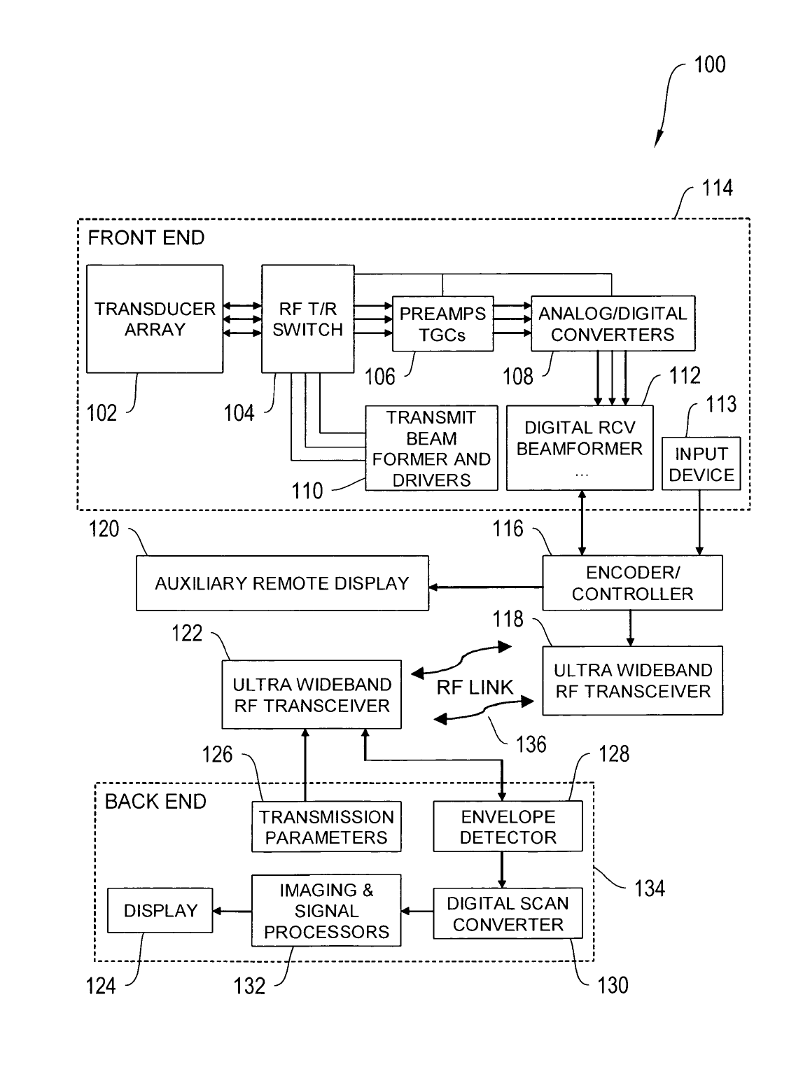

FIG. 1 is a schematic diagram of ultrasound imaging system 100 that operates in accordance with an illustrative embodiment of the invention. The system includes front end 114, a transducer array 102, an input device 113, encoder/controller 116, front end UltraWideBand transceiver 118, which can transfer information to back end UltraWideBand transceiver 122 using a peer-to-peer communication protocol, and back end 134.

UltraWideBand (“UWB”), as discussed in this application, does not refer to a specific technology, but instead refers to an available signal frequency spectrum from approximately 3.1 GHz to approximately 10.6 GHz. UWB can be implemented by using either multiband orthogonal frequency division multiplexing (“MB-OFDM”) technology or direct sequence technology. UWB is significantly different from conventional narrowband radio frequency (RF) and spread spectrum technologies (SS), such as Bluetooth Technology and 802.11a/g. The wide frequency band allocated to UWB can allow a very high data transfer rate, which can exceed 1 Gigabyte per second in some embodiments. The data transfer performance depends upon the specific implementation of UWB. The potential data rate over a given RF link is proportional to the bandwidth of the channel and the logarithm of the signal-to-noise ratio (Shannon's Law). UWB has low power levels, despite its relatively high data transfer rate, and, as a result, UWB signals are expected not to cause interference with other signals. Through signal processing methods such as time-hopping and spectrum spreading, UWB provides a means of multiple-user access without interference. UWB also provides a means for accurate localization of transmitters to develop location-aware networking.

In some embodiments, front end 114 can include transducer array 102, which can transmit and receive sound waves, and can include any known ultrasound transducer array (e.g., a piezoelectric transducer). The transducer operates either in a “transmit” mode or a “receive” mode. When in transmit mode, the transducer generates a short duration acoustic field with a desired center frequency and travels in a specific direction that can be partially reflected when it impacts the scanned tissue. When in receive mode, the reflected sound wave is received by transducer array 102, which can generate ultrasound data corresponding to the reflected sound wave. In some embodiments, transducer array 102 can be external to front end 114, and can be connected to front end 114 by a cable. In other embodiments the front end is integrated with the transducer into one housing.

Front end 114 can include RF transmit/receive switch 104, which generally controls whether transducer array 102 is in transmit mode (i.e., is transmitting sound waves) or receive mode (i.e., is receiving sound waves), and can be coupled to transducer array 102. Front end 114 also contains transmit beamformer and driving circuits 110 for the individual transducer elements when in transmit mode. Transmit beamformer and driving circuits 110 control the signal properties of sound waves transmitted by transducer array 102 when in transmit mode, and can include either a fixed beamformer or an adaptive beamformer.

Front end 114 also includes low noise amplifier and Time Gain Control (“TGC”) circuitry 106 for the individual transducer elements when in receive mode and analog-to-digital converters 108. These components can be used to perform some preprocessing of the received ultrasound data before the ultrasound data is processed by beamformer 112. Front end 114 also includes beamformer 112, which can process the received ultrasound data to determine the direction of maximum signal strength, which can be later used to generate an image from the ultrasound data. Beamformer 112 can in some embodiments be a digital receiving beamformer. In alternative implementations, beamformer 112 is an analog beamformer and there are no analog/digital converters 108.

After it is processed by the beamformer, the ultrasound data can be further processed by encoder/controller 116. In one embodiment, the encoder/controller 116 is implemented in an application specific integrated circuit or in a field programmable gate array. Alternatively, the encoder/controller 116 may be implemented as software for execution on a general purpose processor or digital signal processor. Encoder/controller 116 camencode the ultrasound data before it is wirelessly transmitted to back end UWB transceiver 122 (e.g., using channel coding, frequency interleaving, and/or time interleaving). Encoding the ultrasound data can be advantageous because it can protect the integrity of the ultrasound data during transmission, even in the presence of noise.

After being processed by beamformer 112 and encoder/controller 116, the ultrasound data are transmitted wirelessly from front end UWB transceiver 118 to back end UWB transceiver 122 using wireless UWB connection 136 that uses a peer-to-peer communication protocol. The peer-to-peer communication protocol can, in some embodiments, be an IEEE 1394-conforming protocol (also known as FIREWIRE). In these embodiments, the physical layer can be based on the ECMA-368 (UWB) standard, and the data link layer can utilize an IEEE 1394-conforming protocol adaptation layer. Using a peer-to-peer communication protocol can be advantageous because it can allow for bidirectional communication between front end UWB transceiver 118 and back end UWB transceiver 122. A peer-to-peer communication protocol can also be advantageous because it can allow different front end UWB transceivers to be used interchangeably with back end UWB transceiver 122. The ultrasound data can be transferred in a format suitable either for single element transducers or for a 1-dimensional or 2-dimensional array of transducers.

Data transfer rate for UWB transmission 136 can vary from 100 megabytes per second to 1 gigabyte per second in some embodiments. Uncompressed ultrasound data, sampled at 30 MHz in quadrature and using 16 data bits can require a data transfer rate of approximately 900 megabytes per second. The actual data transfer rate, however, can be less in some embodiments, where UWB transmission does not occur 100% of the time. For example, in a system where the scan depth is 10 cm, the RF lines can have a duration of approximately 133 μs. If, for example, a frame includes 128 lines, UWB signals would be generated at a rate of 17 ms/frame. If the frame rate is 30 frames per second, UWB signals would be produced at a rate of approximately 510 ms for every second, for a duty cycle of about 50%. In addition, the ultrasound data can be compressed in some embodiments. There are several compression techniques available, including, for example, linear predictive coding (LPC) followed by storing a remaining error in a reduced word length, which can reduce the required data transfer rate to about 30-40% of that needed for uncompressed ultrasound data. Thus, using a 50% duty cycle and a compression rate of 30% of the original amount of data will reduce the required data transfer rate to about 135 Mbps, which can be done in embodiments of system 100. Also, in embodiments where quadrature transmission is not used, the required data transfer rate can be further reduced.

In some embodiments, an input device, 113, generates commands signals that are applied to the front end UltraWideBand transceiver, whereby a user can enter operational commands or requests for patient data. The input device can be, for example, a speech recognition system, joystick, keyboard of any type, or a computer mouse. Alternatively, an auxiliary display device 120 can embody a touch screen for data and command entry to the encoder/controller 116. The operational commands can relate to general functionality of ultrasound system 100, and can include commands to turn system 100 on or off, selections for various preset scan modes (e.g., different sound wave intensities that correspond to different applications), and the like. These operational commands can be transmitted using wireless UWB connection 136 from front end 114 to back end 134, where they can be processed.

After the RF ultrasound data is received by the back end UWB transceiver 122, it can be transferred to back end 134 for additional processing, in addition to decompression and decryption. Back end 134 can use signal processor 132 to convert the ultrasound data into image data. The system 100 is compatible with multiple modes of operation, including B-mode, color flow mode, and doppler mode ultrasound, and combinations thereof. This can be done using techniques known in the art. For example, back end 134 can process the received ultrasound data using an envelope detector 128, a scan converter 128, and a signal processor 132. Back end 134 may also include a display 124, an input device (not shown in figure), and a storage device (not shown in figure).

The UWB connection 136 can be utilized for transmission of acoustic field generating commands and receive beamforming commands, generated in the back end of the imaging system, including, but not limited to, commands for frame rate, focal point(s), range, active elements, and scan line repeat for Doppler ultrasound.

In some embodiments, UWB connection 136 can be used to transfer streaming video from back end 134 to auxiliary display 120, which is physically separated from back end 134 and can be in communication with front end UWB transceiver 118 and encoder/controller 116. In some preferred embodiments, auxiliary display 120 can be located proximate to front end 114. This configuration can be advantageous because it can permit the operator of system 100 to view the generated ultrasound image without being near back end 134, thereby giving the operator more freedom and flexibility. In some embodiments, auxiliary display 120 can be carried or worn by the operator of system 100 and/or an assistant to the operator.

Alternatively, pre-stored data in the hospital's information management system, for a given patient, be it image data from ultrasound or other image modalities or other pertinent health care data, can be transmitted via the wireless UWB system to a display carried or worn by the operator and/or assistant to the operator.

System 100 is an improvement over conventional ultrasound systems. System 100 can be used in areas contaminated with hazardous medical material, radioactive materials, and/or contagious diseases, which can require a remote viewing capability generally infeasible with cabled ultrasound systems. Furthermore, because system 100 generally has low power consumption, UWB transmission 136 can be effective up to 10 meters, and low power, inexpensive, miniaturized CMOS components can be utilized. In addition, direct short point-to-point RF transmission used with this approach can be used to locate the given front end and front end UWB transceiver. This can be done through fast UWB timing techniques that allow for quick computation and tracking of the relative positions among sensors.

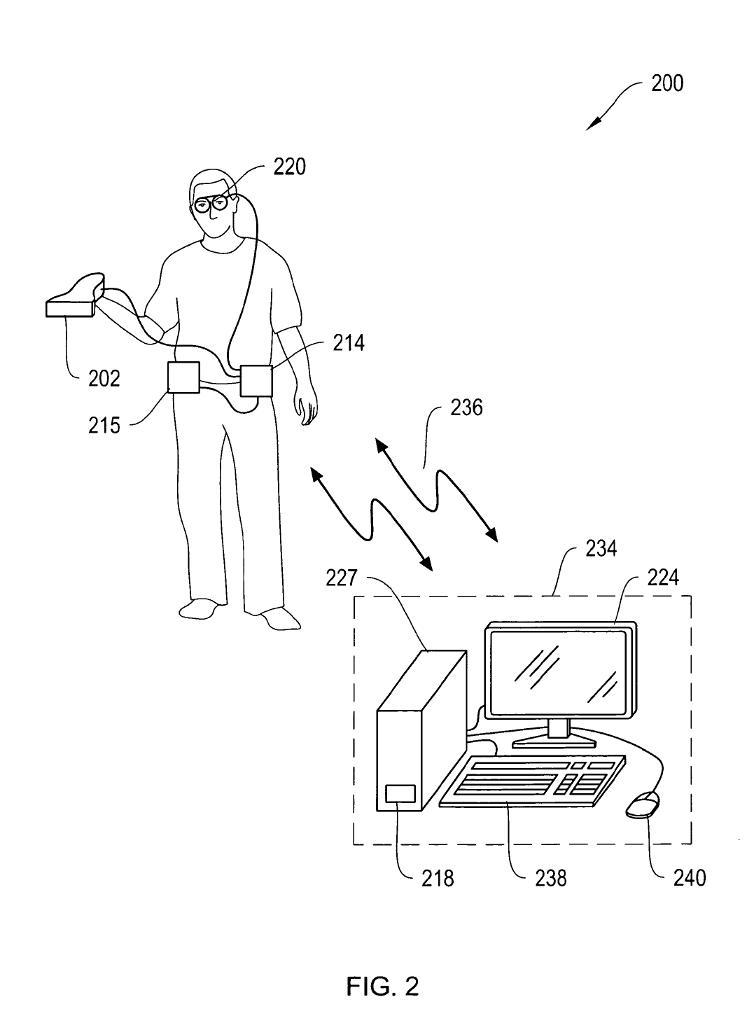

FIG. 2 is a schematic diagram of system 200 for ultrasound imaging according to an illustrative embodiment of the invention. Transducer 202 includes a transducer array, which functions as described above, and can be coupled to front end unit 214 using a wired connection. Front end unit 214 can include not only the elements included in front end 114 described hereinabove, but can also include a front end UWB transceiver (e.g., a transceiver similar to front end UWB transceiver 118 described hereinabove) and an encoder/controller (e.g., an encoder/controller similar to encoder/controller 116 described hereinabove). The front end UWB transceiver within front end unit 214 can be in communication with back end UWB transceiver 218 using wireless UWB connection 236, which uses a peer-to-peer communication protocol, as described hereinabove.

In some embodiments, UWB connection 236 can be used to transfer streaming video from back end 234 to auxiliary display 220, which operates as described hereinabove with reference to auxiliary display 220. In some embodiments, auxiliary display 220 can be a wearable display, and can be head-mounted or worn on the arm of the operator. Using a wearable display can be advantageous because it can enhance the portability of system 200 (i.e., it frees an operator from being required to be proximate to a fixed display). Auxiliary display can be coupled to front end unit 214 using a wired connection. Alternatively, the auxiliary display can be integrated with the back end 234, the UWB transceiver of which can be in wireless communication with the UWB transceiver of the front end.

Power source 215 can be used to supply power to transducer 202, front end unit 214, and auxiliary display 220. Power source 215 can be any device used to power electronic devices (e.g., a battery), and can be coupled to front end 214 unit using a wired connection. Alternatively, the power source can be integrated with the front end or the display.

In an alternative configuration of system 200, front end unit 214 and power source 215 can be included with transducer 202 in a single housing. This configuration can be advantageous because it can further enhance the portability of system 200 (i.e., by reducing the weight of the system), and can free an operator from the use of connecting wires between the individual components. Variations of this alternative configuration, such as an embodiment wherein the front end UWB transceiver is located in a separate housing, for example, are also feasible.

In another alternative configuration of system 200, transducer 202 and front end unit 214 may be included in a single housing, with power source 215 in a separate housing connected to transducer 202 and front end unit 214 using a wired connection. This embodiment can provide enhanced portability for reasons similar to those described hereinabove.

When the operator of the ultrasound system carries a display, either in the form of a head mounted display or a PDA-type display, placed perhaps on the arm, or any other type of carried display, the video information may be transmitted wirelessly in the form of a streaming video from the back end of the ultrasound imaging system to a display carried or worn by the operator. Commands to the back end can be communicated through voice commands, a touch screen type of display 120 or an auxiliary input device housed in the front end unit 114. In one implementation, real time video is streamed over the UWB link in full duplex mode. In alternative implementations, the streaming video is delivered to a wearable display by means of other wireless protocols, such as 802.11. Alternatively, the auxiliary display can be integrated with the back end 234, the UWB transceiver of which can be in wireless communication with the UWB transceiver of the front end.

Furthermore, the UWB transmission protocol may be employed for wireless transmission of vital data versus time for a given patient, such as blood pressure, heart rate, fluid intake, temperature and medication, stored in the hospital's information management system, to a display carried or worn by the operator and/or assistant to the operator. This information may be called up through voice commands, sent over UWB connection 236 from a microphone worn by the operator, to the back-end and then further to the hospital information management system. Alternatively, an input device, such as a touch screen display, can be used to select vital data for display.

In some cases, the display will not be worn, but instead will be permanently mounted in the patient room and will communicate with the hospital's information management system using a wired or wireless link. The display may also be integrated with the back end 234.

System 200 can also include back end 234, which can include back end UWB transceiver 218, processor box 227, display 224, keyboard 238, and mouse 240, and can function as described with respect to back end 134. Back end 234 can take the form of a computer system, as shown in system 200, or any suitable form. Processor box 227 can include an envelope detector, a digital scan converter, a signal processor, and transmission parameters, which all can be implemented either using hardware or software. Keyboard 238 and mouse 240 can serve as input devices to back end 234, thereby allowing an operator to select the operating parameters of system 200. Alternatively, other input devices such as voice commands or touch-screen displays can be used for parameter selection.

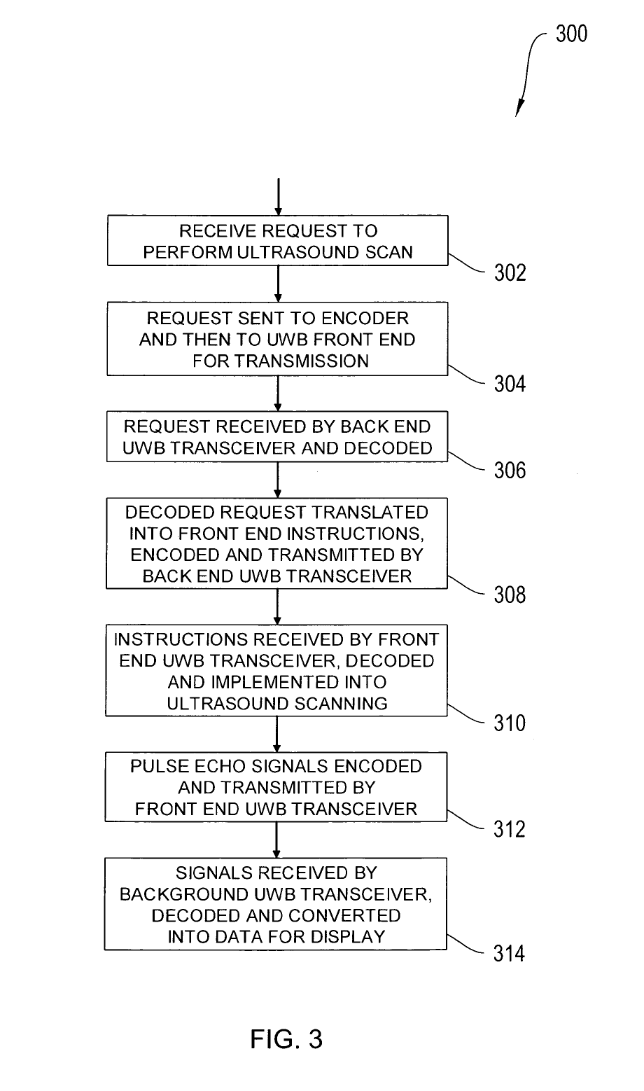

FIG. 3 is a flowchart of method 300 for using an ultrasound imaging system according to an illustrative embodiment of the invention. Method 300 can be applied by a user to systems 100 and 200.

At step 302, a request is received by the ultrasound system to perform an ultrasound scan. At step 304, the request is sent to the encoder and then to the front end UWB transmitter for transmission. At step 306, the request, sent wirelessly, is intercepted by the back end UWB transceiver and decoded. For step 308, the decoded request is translated into front end instructions, encoded and transmitted by the back end UWB transceiver. At step 310, instructions are received by the front end UWB transceiver, decoded and implemented into ultrasound scanning using a transducer, as described hereinabove. At step 312, reflected sound waves are received by the transducer, ultrasound data is generated as described hereinabove within the front end of the ultrasound system and transmitted by the front end UWB transceiver. In some embodiments, the ultrasound data can be processed at step 312. Such processing can include, as described hereinabove, amplification, time gain control processing, analog-digital conversion, and beamforming.

At step 312, the ultrasound data generated at step 310 can be transmitted using UWB from the front end UWB transceiver to the back end UWB transceiver. In step 314, signals are received by the back end UWB transceiver, decoded, and converted into data for display. Finally, at step 314, the ultrasound data is converted into image data or converted into Doppler or other modes. These last steps can be carried out, for example, by using signal processors or entirely in signal processing and scan conversion computer software.

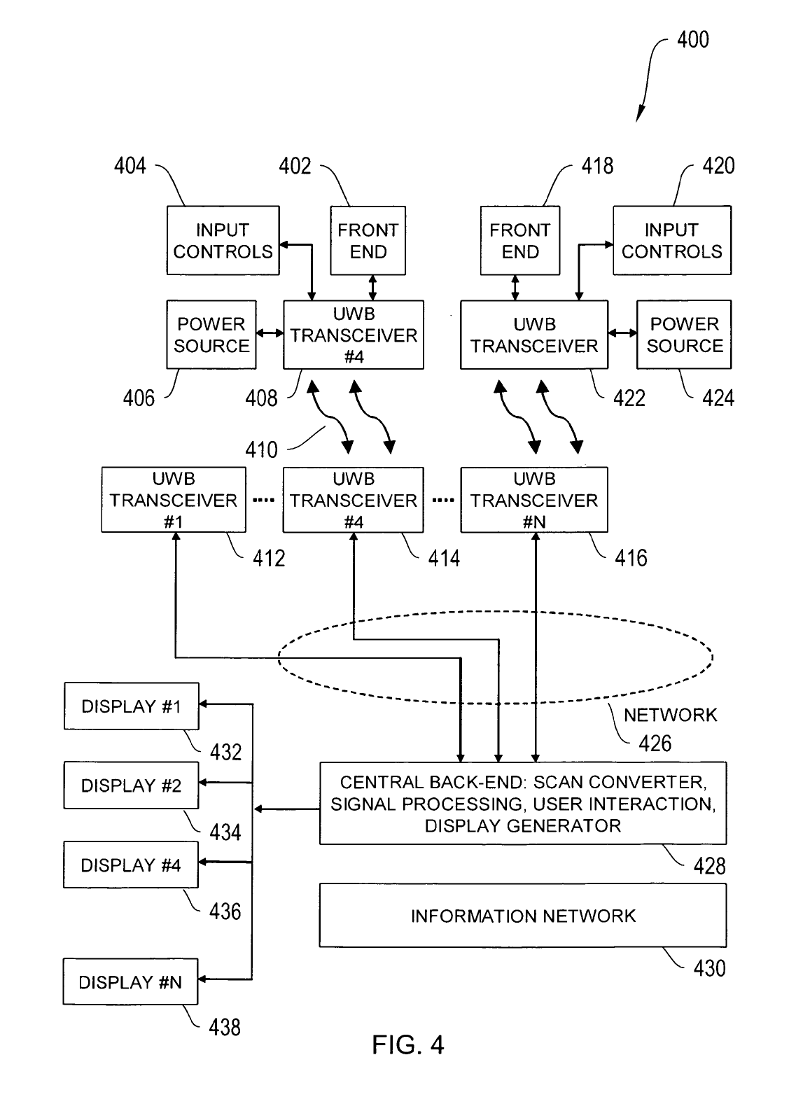

FIG. 4 is a schematic diagram of system 400 for ultrasound imaging according to an illustrative embodiment of the invention. System 400 can be used in applications such as a hospital, wherein it can be desirable to have multiple front end units communicating via UWB to a central computational back end, such that each unit contains one transducer, one input device, one optional display and one front end.

System 400 can include one or more front end units, such as, for example, front end units 402 and 418. Front end units 402 and 418 are generally identical except that they are programmed to have a unique identity code, and function in the same way as their counterparts described in system 100 and system 200. In some embodiments, front end unit 402 can be carried by an operator, and used to communicate with any of back end UWB transceivers 412, 414, and 416 using a UWB connection such as UWB connection 410. Each back end UWB transceiver can receive signals from different front end UWB transceivers by using the multiple access UWB capability; and likewise, each mobile unit can be programmed to recognize many back end transceivers. In addition, using the ranging and locating attributes of UWB, the location of different UWB transceivers can be determined, once accessed.

System 400 can also include one or more back end UWB transceivers, such as, for example, back end UWB transceivers 412, 414, and 416. Back end UWB transceivers 412, 414, and 416 are also generally identical, except that they are programmed to have a unique identity code and function in the same way as back end UWB transceiver 122. In some embodiments, the back end UWB transceivers in system 400 can each correspond to a location within a facility (e.g., a room or a bed in a hospital).

The back end UWB transceivers in system 400 can be in communication with central back end 428 through network 426, which can use any known method to transfer data (e.g., an optical network, a broadband network, and the like). Central back end 428 generally includes the same components and operates in the same manner as back end 134. Central back end 428, however, may be implemented on a substantially more powerful computer, for example, with multiple processor or multiple processing cores, with a larger memory. Central back end 428 can be in communication with information network 430, which can provide an operator of system 400 with patient data, which can include image data.

In some embodiments, central back end 428 can be in communication with information network 430 using a satellite communication system (e.g., any one of the satellite communication systems marketed by Inmarsat P.L.C., Globalstar, Inc., or Intelsat, Ltd.). These embodiments of system 400 can be advantageous, for example, when implemented in rural clinics or remote villages because access can be provided to larger medical facilities for diagnostic support.

Image information, whether real-time ultrasound images or pre-stored images or patient data, can presented to the physician on any of displays 432-438. In some embodiments, displays 432-438 can all be fixed monitors, located in the same general area as corresponding back end UWB transceivers. Because of the limited range of UWB, this usually means that the mobile front end and the nearest back end UWB transceiver are in close proximity. In FIG. 4, a given back end UWB transceiver is associated with a given display, which is placed in the vicinity of that given back end UWB transceiver, so that RF data received by the given UWB transceiver is displayed on its associated display. In some embodiments, displays 432-438 can be portable displays carried by the operator of a front end unit such as front end unit 402. In some embodiments, displays 432-438 can be a mixture of fixed and portable displays. The image data displayed can include real-time ultrasound images, pre-stored images, and/or patient data.

System 400 can allow the doctor to carry out ultrasound scanning in any of the many formats of real-time ultrasound imaging technique known in the art, including 3D imaging, from any location within range of a back end UWB transceiver. In implementations which include a UWB transceiver associated with each bed in a hospital, the central back end can identify the patient being scanned, simply due to the location of the UWB back end transceiver receiving the ultrasound data. Alternatively, the central back end can receive location information about the nearest mobile front end from a receiving UWB transceiver's ranging and locating capability.

There is another significant advantage to the centralized back end computer: it becomes cost-effective to allocate large computational resources to tasks that are typically unavailable for real-time use, such as 3D image reconstruction. A centralized back end computer may also store a large database of clinical information, which can enable a more accurate diagnosis. A centralized back end computer also enables image fusion with previously stored images, including other ultrasound images, as well as CT and MRI images. The design can provide better access to pre-stored patient information, such as ultrasound, CT or MRI images, and data such as medication, temperature, HR, blood pressure.

In an alternative embodiment, one or more of the locations has a complete back end system that includes a back end UWB transceiver, a back end (e.g., executing on a local general or special purpose computer), and a display. The location-specific back ends are further connected to the hospital's information management system. Thus, image processing can be carried out locally, limiting any burden on a hospital's network bandwidth without requiring a central back end, while still providing the local back end access to other information accessible via a hospital network, such as historic patient information, operating room schedules, etc.

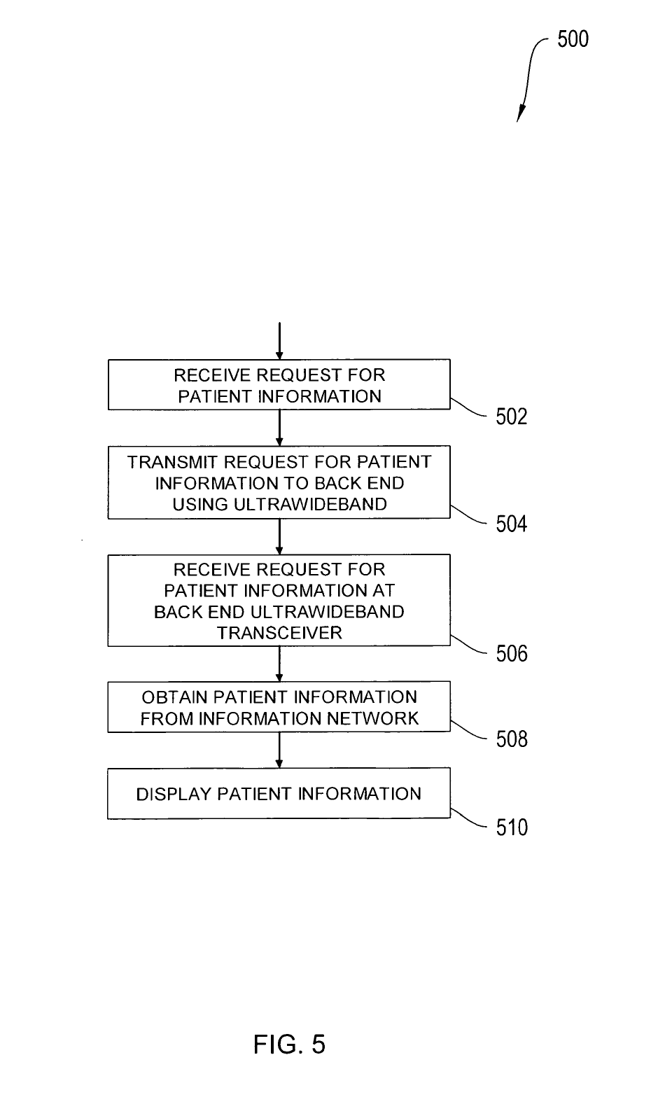

FIG. 5 is a flowchart of method 500 for retrieving patient information from an information network according to an illustrative embodiment of the invention.

At step 502, the ultrasound imaging system receives a request for patient information. In some embodiments, this can be done using an input device at the front end of the imaging system as described herein. Inputting such commands into the front end and its processing of such commands can be carried in a similar fashion as described above in relation to FIG. 3 with regard to the inputting and processing of ultrasound scan commands. The request can be transmitted using a UWB connection, which can use a peer-to-peer communication protocol, to the back end of the system at step 504. At step 506, the back end of the system can receive the request, as described hereinabove. At step 508, the patient information can be obtained from an information network, which includes patient information stored on one or more remote computer systems. At step 510, the patient information can be displayed on a local display. In some embodiments, the patient information can be sent over a UWB connection to the front end, where it can be displayed on an auxiliary display. Alternatively, it can be displayed on a display integrated into or otherwise in communication with the back end.

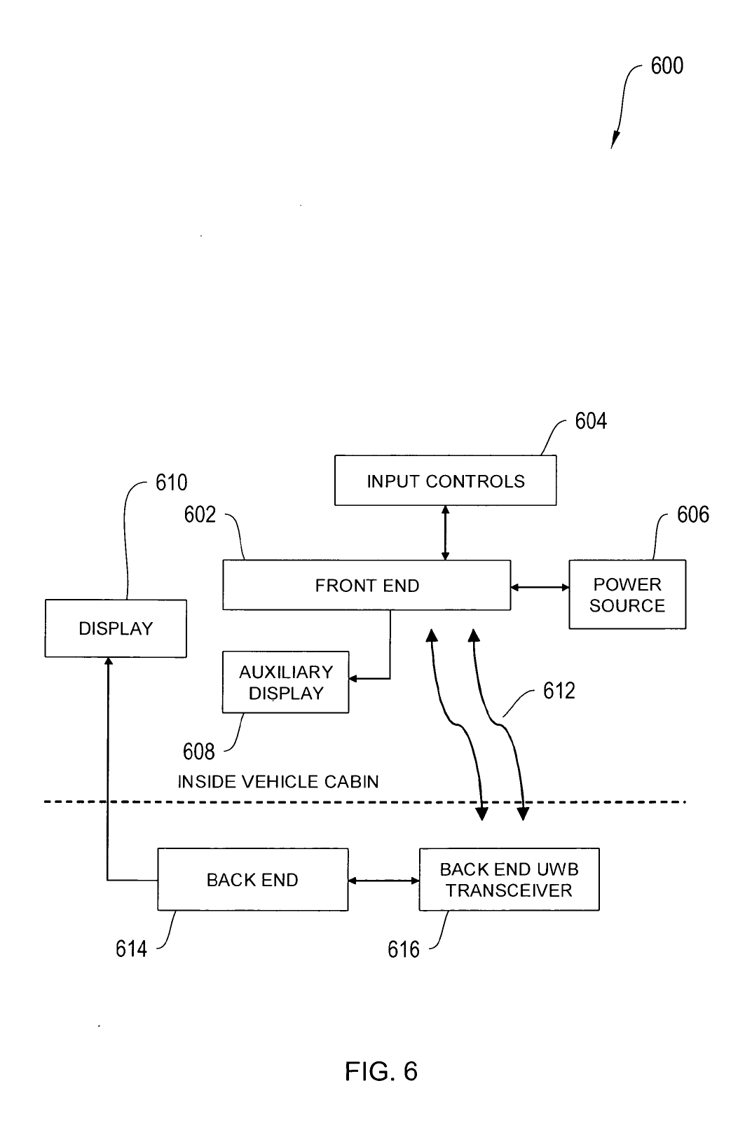

FIG. 6 is a schematic diagram of system 600 for ultrasound imaging in a mobile vehicle application according to an illustrative embodiment of the invention. System 600 can be included in any mobile vehicle, which can include an ambulance, helicopter, or aquatic rescue vessel, where it can be desirable to utilize an ultrasound imaging system. The mobile vehicle in system 600 is divided into a cabin area, which can include display 610, front end unit 602, input controls 604, power source 606, and auxiliary display 608, and the remainder of the vehicle, which can include back end UWB transceiver 616 and back end 614. Locating back end UWB transceiver 616 and back end 614 outside of the vehicle cabin can be advantageous because space can be conserved within the vehicle cabin, which can hold injured patients. Back end 614 and back end UWB transceiver 616 can be located anywhere within the mobile vehicle provided that back end UWB transceiver 616 is within range of front end unit 602 for UWB connection 612 to permit transfer of data between back end UWB transceiver 616 and front end unit 602.

System 600 can include front end unit 602, which can function in the same manner as front end unit 214 and include a front end UWB transceiver in the same housing as the front end. System 600 can also include input controls 604, power source 606, and auxiliary display 608, which can each be in communication with front end unit 602. Input controls 604 and power source 606 function in the same manner as input controls 404 and power source 406 in system 400. Auxiliary display 608 functions in the same manner as auxiliary displays 120 and 220.

Although in system 600, front end unit 602, input controls 604, power source 606, and auxiliary display 608 are all located within the vehicle cabin, this configuration is not necessary. Any or all of front end unit 602, input controls 604, power source 606, and auxiliary display 608 can be removed to a location outside of the mobile vehicle in some embodiments, provided that back end UWB transceiver 616 is within range of front end unit 602 for UWB connection 612 to permit transfer of data between back end UWB transceiver 616 and front end unit 602. This can be advantageous, for example, because ultrasound imaging and diagnosis can be performed on trauma victims who may have spinal injuries or otherwise have conditions that render diagnosis in the vehicle cabin unfeasible.

Front end unit 602 can be in communication with back end UWB transceiver 616 and transmit ultrasound data to back end UWB transceiver 616 using UWB connection 612, as described hereinabove. The ultrasound data can be transferred from back end UWB transceiver 616 to back end 614, which functions in the same manner as back end 134. Image data resulting from the processing of the ultrasound data can be transferred from back end 614 to display 610 and/or to auxiliary display 608 as described hereinabove.

In some embodiments, back end 614 can be in communication with an information network (e.g., a satellite communication system or other wireless communication system) as described hereinabove with respect to system 400. Such embodiments can be advantageous because, for example, assistance in diagnosis of injured persons can be obtained from an emergency department or the surgical facilities of the receiving hospital.

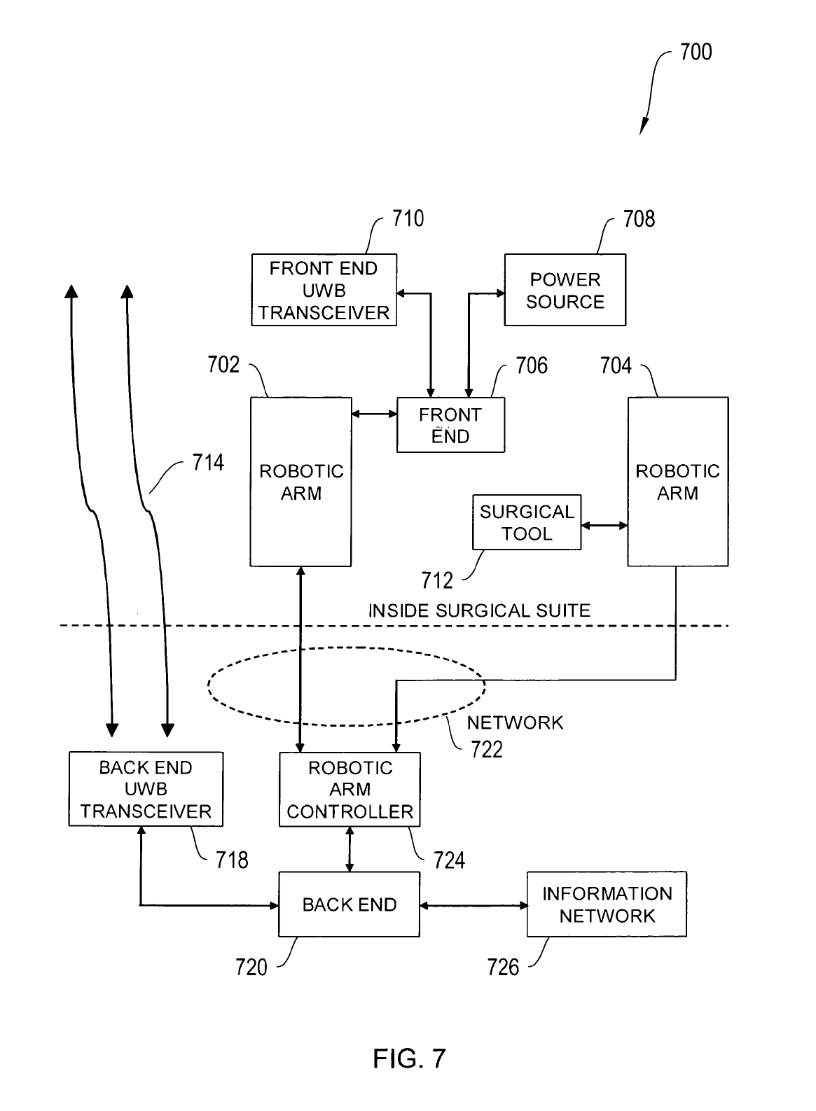

FIG. 7 is a schematic diagram of system 700 for remotely guided surgery in a surgical suite using ultrasound imaging according to an illustrative embodiment of the invention.

System 700 can be included in a surgical suite. Front end 706, which functions in the same manner as front end 114, can be used to generate ultrasound data as described hereinabove. Through UWB connection 714, the ultrasound data can be transferred from front end UWB transceiver, which can receive the ultrasound data from front end 706, to back end UWB transceiver, which is outside of the surgical suite. The ultrasound data can be transferred to back end 720, which functions in the same manner as back end 134. In some embodiments, back end 720 is in communication with information network 726, and can retrieve patient information as described in FIG. 4 and the accompanying text.

By locating back end UWB transceiver 718 and back end 720, which are generally larger than front end 706, front end UWB transceiver 710 and power source 708, outside of the surgical suite, valuable space can be saved within the surgical suite. Additionally, locating back end UWB transceiver 718 and back end 720 outside of the surgical suite can remove one additional source of contamination. Moreover, embodiments using voice commands or sealed touch screen to guide system 700 gives convenient control of the ultrasound system and removes the keyboard that could easily be contaminated with fluids and is in itself a source of contamination.

System 700 can be used for remotely guided surgery. Front end 706 can be manipulated by robotic arm 702, which can position front end 706 to generate ultrasound data as described hereinabove. Robotic arm 702 can be manipulated from outside of the surgical suite using robotic arm controller 724, which can be connected to robotic arm 702 and robotic arm 704 using network 722. Network 722 can be any suitable data transmission network, and can be implemented using fiber optic cables in some embodiments.

Robotic arm controller 724 can also be used to control robotic arm 740, which can manipulate surgical tool 712 to perform a desired surgical procedure. Surgical tool 712 can be an apparatus that can be used to perform remote surgery, and can include bladed cutting instruments, instruments that use lasers or other types of radiation, high intensity focused ultrasound and/or clamping instruments. In some embodiments, multiple robotic arms can be used to perform the remotely guided surgery, and these may be controlled by multiple robotic arm controllers or a single robotic arm controller. System 700 can be advantageous when used for remotely guided surgery because it can reduce the number of interfering wires in the surgical suite, reduce the number of devices in the surgical suite (thereby conserving space and reducing sources of contamination).

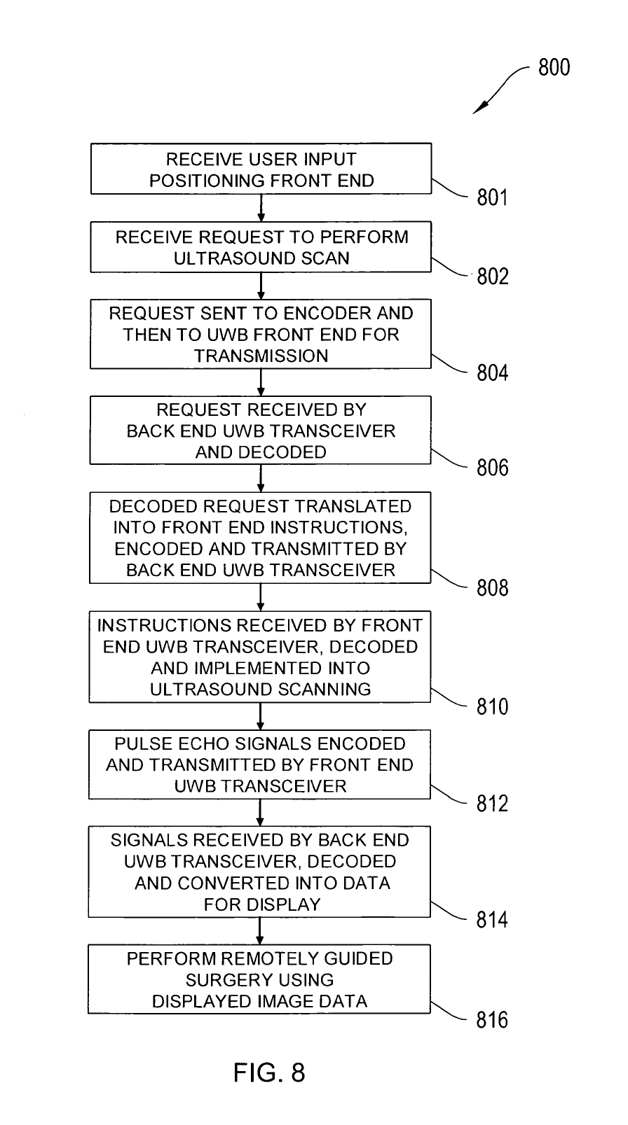

FIG. 8 is a flowchart of method 800 to perform remotely guided surgery using an ultrasound imaging system according to an illustrative embodiment of the invention. At step 801, the system for performing remotely guided surgery can receive user input for positioning the front end of the ultrasound imaging system, which functions as described hereinabove. The user input can be processed by an encoder, which causes the front end to be positioned accordingly. The encoder may be connected to the back end, or it may be a separate device in communication with the back end. Steps 802-814 are the same as steps 302-314, resulting in image data that corresponds to the ultrasound data generated at steps 810 and 812. At step 814, the system can display the image data as described hereinabove. Based upon the image data, a surgeon can determine how to perform the remotely guided surgery. At step 816, the system can receive user input directing the remotely guided surgery, and perform the surgery accordingly as described hereinabove. These steps can be repeated interactively to guide the surgery in real-time.

As noted above, the order in which the steps of the methods described hereinabove are performed is purely illustrative in nature. In fact, the steps can be performed in any order or in parallel, unless otherwise indicated by the present disclosure. Furthermore, the invention may be embodied in other specific forms without departing from the spirit or essential characteristics thereof. The forgoing embodiments are therefore to be considered in all respects illustrative, rather than limiting of the invention.

What is claimed is:

1. An ultrasound system, the system comprising:
a transducer array;
a front end, the front end comprising a beamformer and a front end UltraWideBand transceiver coupled to the beamformer, the beamformer providing ultrasound data;
a back end physically separated from the front end comprising a back end UltraWideBand transceiver for receiving ultrasound data from the front end UltraWideBand transceiver, and a signal processor circuit for converting received ultrasound data from the front end into image data for display via a peer-to-peer communication protocol, the ultrasound data.

2. The ultrasound system of claim 1, wherein the front end UltraWideBand transceiver and the back end UltraWideBand transceiver are in communication using an UltraWideBand protocol in duplex mode.

3. The ultrasound system of claim 1, wherein the front end UltraWideBand transceiver and the back end UltraWideBand transceiver are in communication using an UltraWideBand protocol in full duplex mode.

4. The ultrasound system of claim 1, wherein the peer-to peer communication protocol comprises an IEEE 1394-conforming protocol.

5. The ultrasound system of claim 1, wherein the front end comprises a front end housing holding the beamformer and the front end UltraWideBand transceiver couples to the front end housing via a cable.

6. The ultrasound system of claim 1, wherein the front end comprises a front end housing holding the beamformer and the front end UltraWideBand transceiver.

7. The ultrasound system of claim 1, wherein the front end UltraWideBand transceiver and the back end UltraWideBand transceiver are configured to utilize ECMA-368 UltraWideBand.

8. The ultrasound system of claim 1, further comprising a display, which is physically separated from the back end while in communication with the back end.

9. The ultrasound system of claim 8, wherein the display is wearable.

10. The ultrasound system of claim 8, wherein the back end UltraWideBand transceiver is in communication with a computer network, the computer network storing a plurality of patient data, and wherein the back end UltraWideBand transceiver is configured to transmit a selection of the patient data from the computer network to the display.

11. The ultrasound system of claim 10, wherein the selected patient data comprises a set of stored patient image data.

12. The ultrasound system of claim 10, wherein the selected patient data comprises a set of vital data versus time for a patient, the set of vital data versus time selected from the group consisting of blood pressure, heart rate, fluid intake, temperature, and medication.

13. The ultrasound system of claim 1, wherein the beamformer comprises an analog beamformer.

14. The ultrasound system of claim 1, wherein the beamformer comprises a digital beamformer.

15. The ultrasound system of claim 1, wherein the front end UltraWideBand transceiver and the back end UltraWideBand transceiver are configured to transmit and receive data in the form of RF beamformed data.

16. The ultrasound system of claim 1, wherein the back end is implemented on a local computer.

17. The ultrasound system of claim 16, wherein the local computer is configured to access a set of patient data stored at a remote location.

18. The ultrasound system of claim 1, further comprising a display centrally mounted in a surgical suite in communication with the back end for displaying the image data.

19. The ultrasound system of claim 1, further comprising a control apparatus for accepting commands for controlling the ultrasound system.

20. The ultrasound system of claim 19, wherein i) the control apparatus is coupled to the front end and in communication with the back end to transmit imaging commands thereto, ii) the front end UltraWideBand transceiver is configured to transmit the imaging commands to the back end UltraWideBand transceiver, iii) the back end is configured to convert the imaging commands into a set of instructions for the front end, and iv) the back end UltraWideBand transceiver is configured to transmit the set of instructions to the front end through the front end UltraWideBand transceiver.

21. The ultrasound system of claim 19, wherein the control apparatus is coupled to the back end and is in communication with the front end, and the back end UltraWideBand transceiver is configured to transmit acoustic field generating commands to the front end through the front end UltraWideBand transceiver.

22. The ultrasound system of claim 21, wherein the front end includes a processor for translating the commands into instructions for ultrasound scanning.

23. The ultrasound system of claim 19, wherein the control apparatus comprises a microphone, the microphone being configured to accept voice commands to control the ultrasound system.

24. The ultrasound system of claim 1, further comprising an encryption module for encrypting data transferred by at least one of the front end and back end UltraWideBand transceivers.

25. An ultrawideband system, comprising:
a plurality of locations;
a display located in each respective location; and
at least one back end in communication with one or more front ends physically separated from the back end,
wherein each of the one or more front ends comprises:
a beamformer for producing ultrasound data corresponding to a selected display; and
a front end UltraWideBand transceiver coupled to the beamformer for transmitting the produced ultrasound data provided by the beamformer to the back end via a peer-to-peer communication protocol; and
wherein the back end comprises:
a signal processor circuit for generating image data by converting received ultrasound data into image data; and
a back end UltraWideBand transceiver for i) receiving ultrasound data from a front end UltraWideBand transceiver of one of the front ends, and ii) transmitting the image data generated by the signal processor circuit from the received ultrasound data to the front end UltraWideBand transceiver of the front end from which the corresponding ultrasound data was received via the peer-to-peer communication protocol.
